(19) United States
(12) Patent Application Publication
(10) Pub. No.: US 2025/0321219 A1
(43) Pub. Date: Oct. 16, 2025

(54) Devices For Image-Based Analysis Of Waste Material Within A Canister

(71) Applicant: Stryker Corporation, Portage, MI (US)

(72) Inventors: Steven D. Scherf, Oakland, CA (US); Matthew B. Pastrick, Kalamazoo, MI (US); Christopher J. Keizer, Hudsonville, MI (US); Helayna A. Barrett, Kalamazoo, MI (US); Michael A. Stinchcomb, Spring Lake, MI (US); Benjamin Edinger, Grand Haven, MI (US); Jeffrey Conway, Incline Village, NV (US); Russell Conway, Capitola, CA (US)

(73) Assignee: Stryker Corporation, Portage, MI (US)

(21) Appl. No.: 18/871,890

(22) PCT Filed: Jun. 8, 2023
(86) PCT No.: PCT/US2023/024800

§ 371 Date: Dec. 5, 2024

Related U.S. Application Data
(60) Provisional application No. 63/350,217, filed on Jun. 8, 2022.

(51) Int. Cl. G01N 33/49; A61M 1/00; G06K 7/14

(52) U.S. Cl. CPC G01N 33/49; A61M 1/60; G06K 7/1417; A61M 2205/3306; A61M 2205/6036; A61M 2205/6063

(57) ABSTRACT

Devices for facilitating image-based determination of a blood component in medical waste. The device may be an insert including means for positioning and supporting the insert within the canister for an imaging feature of the insert to be spaced apart from an inner surface of the canister. The insert may include struts configured to engage ribs of the canister in a press-fit arrangement. The struts may be tapered inwardly toward a base of the canister. The insert may define slots providing fluid communication between a rear side and a front side of the insert. The insert may include a frame configured to be compressed to resiliently deform, and engage the canister in a press-fit arrangement. The insert may include feet for providing a fulcrum to facilitate coupling surfaces of the insert to the inner surface with adhesive.

### PRIORITY CLAIM

This application claims priority to and all the benefits of U.S. Provisional Patent Application No. 63/350,217, filed Jun. 8, 2022, the entire contents of which are hereby incorporated by reference.

### BACKGROUND

A byproduct of surgical procedures is the generation of liquid, semisolid, and/or solid waste material. The medical waste may be removed from the surgical site through a suction tube under the influence of a vacuum. The medical waste may include liquids such as blood, interstitial fluid, mucus, irrigating fluid, and the like.

Determining blood loss during surgery may be used to monitor intraoperative patient health. Advances in imaging and computing have provided for quantifying blood loss by capturing an image of the fluid-containing media, such as a canister. One such system is sold under the tradename Triton by Gauss Surgical, Inc. (Menlo Park, Calif.) and disclosed in commonly-owned U.S. Pat. No. 9,773,320, issued Sep. 26, 2017, the entire contents of which are hereby incorporated by reference. The system includes a canister within which an insert is disposed and configured to permit a thin layer of fluid to be situated between the insert and an inner surface of the canister.

The insert may be separately provided from the canister, and therefore it may be necessary for a surgical technician or another individual to prepare the canister by coupling the insert with the canister in an instructed manner in advance of the procedure. While the canister and the insert may include complementary geometries to facilitate accurate positioning of the insert, the process remains prone to human error, and small deviations in the position of the insert may implicate the accuracy of the image-based processing. Therefore, there is a need in the art to address the aforementioned technical challenge.

### SUMMARY

The present disclosure is directed to devices to be disposed within a canister for image-based analysis of waste material. The devices may in the form of an insert or other similar structure. The insert includes several geometries, at least one of which is spaced apart from an inner surface of the canister to define a gap. The gap is of a known and/or fixed distance. The insert generally includes a front side opposite a rear side. An imaging feature is on the front side of the insert includes an imaging surface, and optionally a second imaging surface. The imaging feature generally includes a first end and a second end opposite the first end with the imaging surface(s) extending between the first and second ends. The first imaging surface and the second imaging surface may be positioned lateral to one another in a side-by-side arrangement. The second imaging surface is recessed from the front surface, and may be separated from one another by a ridge. The first imaging surface is spaced apart from the inner surface by a first distance and the second imaging surface is spaced apart from the inner surface by a second distance. The second distance may be greater than the first distance.

An upper aspect may be positioned above the imaging feature and include a front surface. The front surface may be contoured to the inner surface of the canister. The imaging feature may be recessed from the front surface of the upper aspect. An edge may separate the imaging feature from the upper aspect. The upper aspect may be sized to be at least equal to a size and shape of a reference marker. The reference marker facilitates locating the region of the image associated with the imaging feature and analysis thereof, and further provides for color-correction to compensate for variances in ambient lighting or other optical aberrations. The reference marker may be affixed to the outer surface of the canister, or affixed to the upper aspect of the insert.

The insert may include at least one projection extending forward from the imaging surface, the front surface, or another surface of the insert. There may be one, two, or three or more projections. An exemplary implementation includes the imaging feature being generally square or rectangular in shape with the each of four projections disposed adjacent to a respective one of four corners of the imaging feature. The projections are configured to separate the imaging surface from the inner surface of the canister by a gap.

The insert may include means for supporting the insert within the canister to locate and maintain a position the imaging feature. A first implementation utilizes a press-fit design. The insert includes a frame extending rearwardly from the imaging feature. The frame may include legs each formed from arcuate members of the frame. Each of the legs may include a foot contoured or angled relative to the arcuate members. A second implementation includes the insert being generally rectangular in shape with a height greater than a width. The width of the insert is sized to be snugly secured between adjacent ribs formed within the canister. The insert may include struts defining slots between the struts. The struts facilitate the press-fit interaction with the ribs. The front surface and the imaging feature may be disposed between the struts. In another implementation, the insert is affixed to the canister with adhesive. The adhesive may be affixed to the front surface, to a lower front surface positioned opposite the imaging feature, or another surface of the insert.

The slots may extend between the front and rear sides. The slots provide fluid communication from the rear side to the front side of the insert. A lower edge of the insert may define a lower vent. A handle or grip may be a flange extending rearwardly from the front surface or another structure of the insert. The insert may include an alignment configured to provide a visual reference to the user when affixing the reference marker to the upper aspect, or to the exterior of the canister. The alignment feature may be a dimple formed on or within the front surface adjacent to the imaging feature. The alignment feature may be an aperture extending through the insert. Indicia, such as a numerical value indicative of the distance of the gap between the imaging feature and the inner surface of the canister, may be provided on the insert.

Therefore, according to certain inventive aspects of the present disclosure, the insert includes a front side opposite a rear side, and an imaging feature disposed on the front side. The imaging feature comprises at least one imaging surface configured to be spaced apart from an inner surface of the canister by a gap. A first strut is spaced apart from a first end of the imaging feature to define a first slot therebetween, and a second strut is spaced apart from a second end of the imaging feature to define a second slot therebetween. The first and second struts are configured to engage ribs of the canister to secure the insert within the canister. The first and second slots extend between the front side and the rear side and are configured to provide fluid communication between the gap and a remaining volume of the canister.

According to certain inventive aspects of the present disclosure, the insert includes a front side opposite a rear side, and an imaging surface disposed on the front side. At least three projections extend forward from the front side and configured to engage an inner surface of the canister to maintain a gap between the imaging surface and the inner surface of the canister.

According to certain inventive aspects of the present disclosure, the insert includes a front side opposite a rear side, and an imaging feature disposed on the front side. The imaging feature includes a first imaging surface, and a second imaging surface arranged side-by-side to the first imaging surface. Each of a plurality of projections extends from the front side and configured to engage an inner surface of the canister to maintain a first gap between the first imaging surface and the inner surface of the canister, and a second gap between the second imaging surface and the inner surface. The first gap is different than the second gap.

According to certain inventive aspects of the present disclosure, the insert includes a front side opposite a rear side, and an imaging feature disposed on the front side. The imaging feature is configured to be spaced apart from an inner surface of the canister by a gap. An aperture is defined between the front side and the rear side. An upper aspect positioned above the imaging feature. The aperture provides an alignment feature configured to be aligned with a complementary geometry of a reference marker.

In certain implementations, an alignment feature may be positioned adjacent to an edge separating the upper aspect and the imaging feature. The alignment feature configured to be aligned with a complementary geometry of the reference marker. The alignment feature may be an aperture extending between the front side and the rear side. The aperture is configured to permit fluid to flow between the front side and the rear side of the imaging feature. The aperture may be along a midpoint of a width of the upper edge of the imaging feature.

In certain implementations, a handle may extend rearwardly from the upper aspect and configured to require insertion of the insert to within the canister in a single orientation. The first and second struts may each be tapered inwardly downward so as to be engageable with the ribs of the canister in the single orientation. The first and second struts may extend below a lower edge of the imaging feature so as to define the lower vent between the imaging feature and a lower surface of the canister.

In certain implementations, a frame extends rearwardly from the imaging feature. The frame comprises legs each formed from arcuate members of the frame. The legs are sized such that the insert has an outer dimension approximating an inner diameter of the canister.

### BRIEF DESCRIPTION OF THE DRAWINGS

FIG. 1 depicts an imaging device capturing of an image of a canister for image-based analysis of the waste material contained therein. An insert is disposed within the canister.

FIG. 2A is a front perspective view of a first implementation of the insert.

FIG. 2B is a front elevation view of the insert.

FIG. 2C is a rear perspective view of the insert.

FIG. 2D is a side elevation view of the insert.

FIG. 3A is a front view of a second implementation of the insert disposed within the canister.

FIG. 3B is a front perspective view of the insert.

FIG. 3C is a side elevation view of the insert.

FIG. 3D is a front elevation view of the insert.

FIG. 4A is a front perspective view of a third implementation of the insert.

FIG. 4B is a front elevation view of the insert.

FIG. 4C is a side elevation view of the insert.

FIG. 4D is a rear elevation view of the insert.

FIG. 5A is a front perspective view of a fourth implementation of the insert.

FIG. 5B is a front elevation view of the insert.

FIG. 5C is a side elevation view of the insert.

FIG. 5D is a rear elevation view of the insert.

FIG. 6A is a front perspective view of a fifth implementation of the insert.

FIG. 6B is a rear perspective view of the insert.

### DETAILED DESCRIPTION

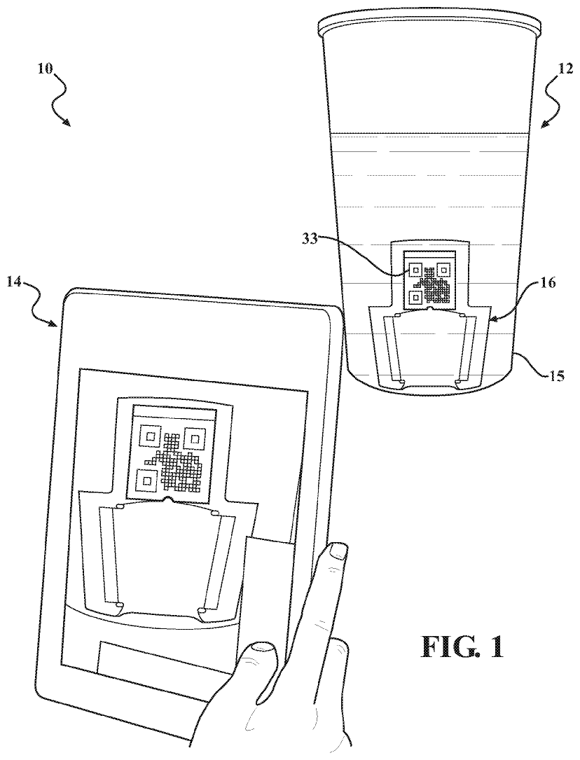

FIG. 1 shows a system 10 for image-based analysis of waste material, and in particular image-based quantification of blood loss within a canister 12. The canister 12 may be freestanding on a surface or coupled to another support structure. The canister 12 includes an inlet and outlet ports (not identified) configured to be coupled to suction lines. The suction line coupled to the outlet port is configured to be arranged in fluid communication with a vacuum source, for example, a medical waste collection system sold under the tradename Neptune by Stryker Corporation (Kalamazoo, Mich.). Alternatively, the vacuum source may be integrated with the medical facility. The suction line coupled to the inlet port is configured to be arranged in fluid communication with a suction instrument to be deployed at a surgical site to draw the medical waste into the canister 12 under the influence of suction provided by the vacuum source. The medical waste is collected within the canister 12.

When desired, a user may capture an image of the canister 12, with the medical waste disposed therein, to be processed by a processor (not shown) executing instructions for determining a proportion or quantity of blood within the canister 12 based on the image. As shown in FIG. 1, the image may be captured by a camera associated with a device 14, such as a tablet or a smartphone. A depth sensor of the device 14 may also be used. Exemplary systems and methods for analyzing the image are disclosed in the aforementioned U.S. Pat. No. 9,773,320, and further disclosed in commonly-owned U.S. Pat. No. 9,171,368, issued Oct. 27, 2015, the entire contents of which are hereby incorporated by reference. For example, certain implementations of image-based determinations include extracting a redness or other color component value in the image to estimate the blood component (e.g., hemoglobin) within the waste material. The volume of the waste material may be inputted or determined, and the volume of blood in the canister 12 is determined.

The deeper red tint may result in color signal saturation that itself may yield suboptimal readings and determinations. Likewise, overly opaque fluids may result from the liquid containing excessive red blood cells with insufficient free hemoglobin, or from lysis of an unknown portion of the whole red blood cells. To ensure consistent and accurate image-based determinations of the blood component, an insert 16 is configured to be disposed within the canister 12. The insert 16 includes several geometries, at least one of which is spaced apart from an inner surface of the canister 12 to define a gap of known and fixed distance. The gap permits a thin layer of fluid to be situated between the insert 16 and the inner surface of the canister 12. Further, the insert 16 may be white or nearly white. With a white backdrop to the thin layer of fluid, the fluid exhibits a region of at least substantially uniform color that is below a color intensity to cause signal saturation. In certain implementations, the insert 16 is formed from a polymer sold under the tradename Lustran® by INEOS Group, Ltd. (London, United Kingdom) or another suitable polymer.

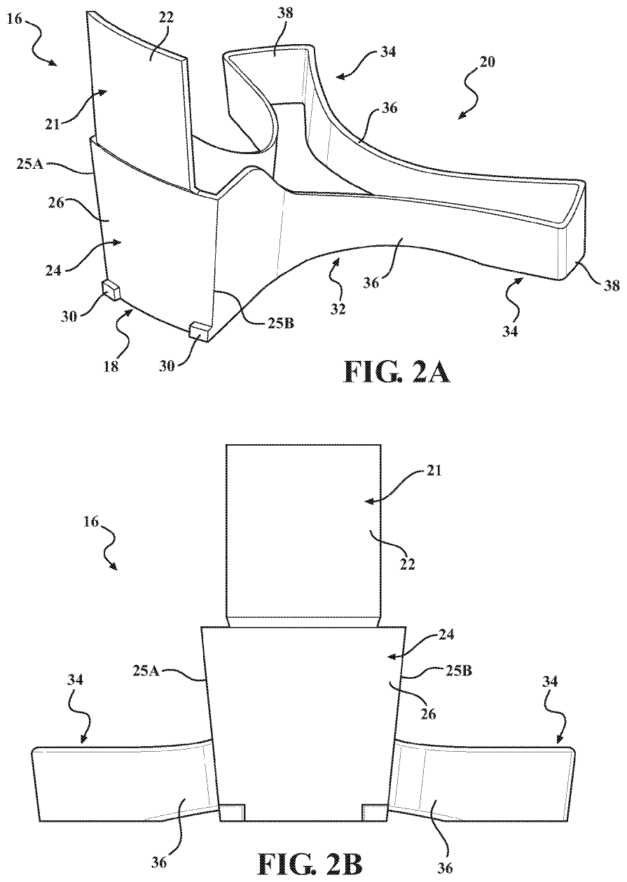

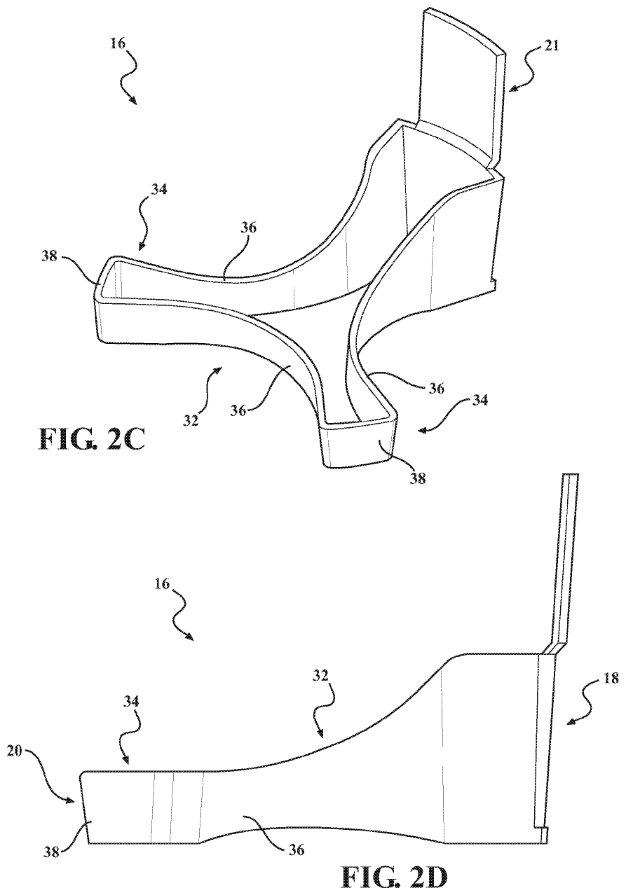

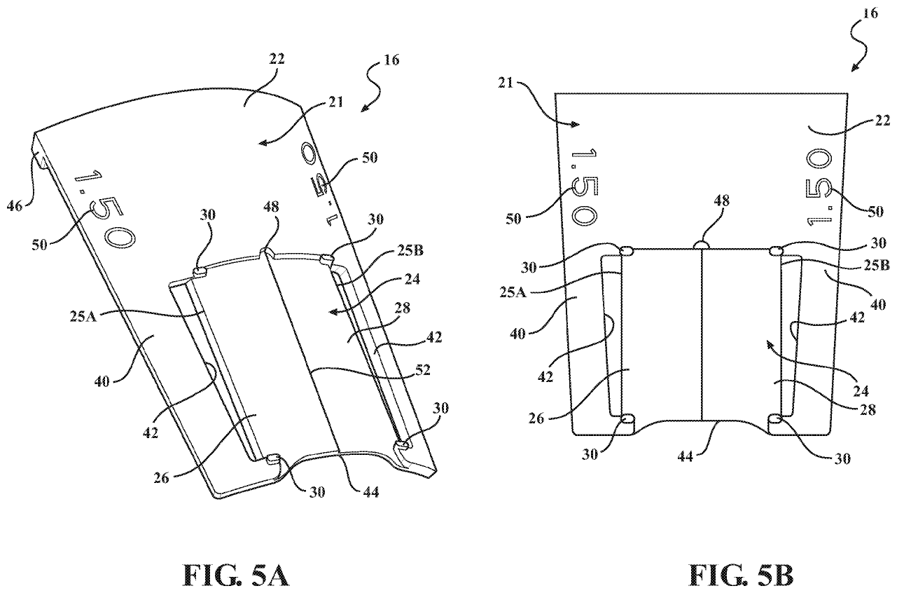

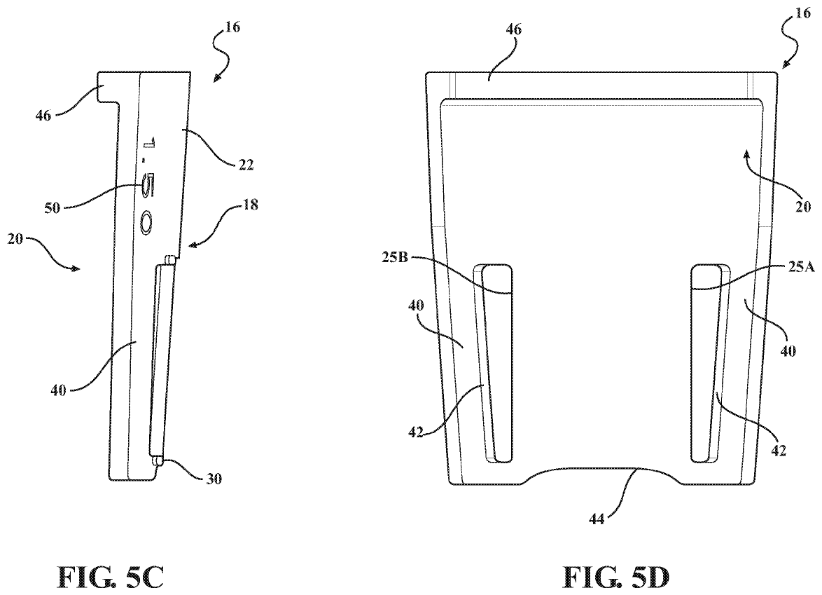

Referring to FIGS. 2A-2D, an implementation of the insert 16 is shown with additional implementations to be later described. The insert 16 generally includes a front side 18 opposite a rear side 20. For convention, the front side 18 is configured to be positioned adjacent or nearer to the inner surface of the canister 12, and the rear side 20 is opposite the front side 18. An upper aspect 21 may include a front surface 22 and may be configured to be positioned adjacent to or in an abutting relationship with the inner surface of the canister 12. An imaging feature 24 of the insert 16 includes an imaging surface 26, and optionally a second imaging surface 28 (see FIGS. 5A and 5B) to provide a gradient of increasing color intensity being below that which may cause the signal saturation. The imaging feature generally includes a first end 25A and a second end 25B opposite the first end 25A with the imaging surface(s) 26 extending between the first and second ends 25A, 25B. The front surface 22 may be contoured to an inner diameter of the inner surface to promote consistent positioning of the imaging feature 24 within the canister 12 and limit inadvertent movement of the insert 16 relative to the canister 12. The upper aspect 21 may be positioned above the imaging feature 24, and the imaging feature 24 may be recessed from the front surface 22 of the upper aspect 21. An edge may separate the imaging feature 26 from the upper aspect 21. With the front surface 22 directly contacting the inner surface of the canister 12, the imaging surface 26 is spaced apart from the inner surface by a known distance. In one example, the distance is 1.7 millimeters. It is more broadly contemplated that the known distance may be within the range of approximately 0.7 to 5.7 millimeters, and more particularly within the range of 1.2 to 3.7 millimeters. The known distance permits a thin layer of fluid to be situated between the imaging surface 26 and the inner surface. The thin layer of fluid exhibits at least substantially uniform color, and the controller is configured to locate the region of the image associated with the imaging feature 24 for the image-based determinations of the blood component.

The upper aspect 21 may be sized to be at least equal to a reference marker 33 (see FIG. 1) configured to be detected by the optical sensor when capturing the image of the canister 12. The reference marker 33 facilitates locating the region of the image associated with the imaging feature 24 and analysis thereof, and further provides for color-correction to compensate for variances in ambient lighting or other optical aberrations. An exemplary implementation of the reference marker 33 is disclosed in commonly-owned U.S. Pat. No. 9,824,441, issued Nov. 21, 2017, the entire contents of which are hereby incorporated by reference, in which a quick response (QR) code is affixed with adhesive to an outer surface of the canister 12 corresponding to a position of the upper aspect 21 of the insert 16 disposed within the canister 12. The QR code may be printed to be red of a known red color component value. The processor may adjust values based on the red color component value as detected in the captured image relative to the known red color component value. In other words, the red color component value of the reference marker 33 may assist the processor with color calibration to improve the accuracy of the optical sensor in quantifying the blood component according to the color of the fluid. Further, having the upper aspect 21 positioned behind and being equal to or larger in size than the reference marker 33 provides for improved image segmentation of the red QR code within the image that otherwise may include dark red tinted fluid in the canister 12. Alternatively, it is contemplated that the reference marker 33 may be affixed to the insert 16, in particular, the upper aspect 21 of the insert 16. The upper aspect 21 may also be configured to abut the inner surface of the canister 12 such that the reference marker 33 affixed to the upper aspect 21 abuts the inner surface of the canister 12. In such a configuration, the abutment between the reference marker 33 and the inner surface of the canister 12 limits fluid ingress between the reference marker 33 and the canister 12. This may be advantageous where the color of the fluid would affect the visibility (e.g., the color) of the reference marker 33 if fluid was present in front of the reference marker 33. Additionally or alternatively, a clear film may be affixed to cover the reference marker 33.

The insert 16 may include at least one projection 30 configured to be positioned adjacent to or in an abutting relationship with the inner surface of the canister 12. As shown in FIGS. 2A and 2B, the projections 30 extend forward from the imaging surface 26. The projections 30 are sized such that the imaging surface 26 may be spaced apart from the inner surface by the known distance along its entire height, also referred to herein as a gap. In other words, the projections 30 may be feet or standoffs and configured to separate the imaging surface 26 from the inner surface of the canister by a gap. For example, the projections 30 may extend forward from the imaging surface 26 by a same distance by which the front surface 22 extends forward from the imaging surface 26. The imaging feature 24 may be positioned between the projections 30 and the upper aspect 21.

The insert 16 may include means for supporting the insert 16 within the canister 12 to locate and maintain a position the imaging feature 24. The means for supporting the insert 16 may couple the insert 16 within the lower portion of the canister 12, or to a sidewall of the canister 12, or combinations thereof. The implementation of FIGS. 2A-2D utilizes a press-fit design to facilitate ease of insertion into the canister 12. The insert 16 includes a frame 32 extending rearwardly from the imaging feature 24. The frame 32 is configured to provide two points of contact in addition to that provided by the front surface 22 and the projections 30. The arrangement may loosely be considered geometrically as a circular canister circumscribing the triangular insert.

The frame 32 may include legs 34 each formed from arcuate members 36 of the frame 32. Each of the legs 34 may include a foot 38 contoured or angled relative to the arcuate members 36 to be flush with and abut the inner surface of the canister 12 to provide the aformentioned points of contact. The legs 34 are sized such that the insert 16 has an outer [circular] dimension slightly greater than an inner diameter of the canister 12. To install the insert 16 into the canister 12, the user may squeeze or compress inwardly on the arcuate members 36 (see arrows in FIG. 2C). Owing the resiliently flexible material from which the insert 16 is formed, there is slight resilient deformation for the insert 16 to be fully seated on the base 15 of the canister 12. After suitably positioned, the compressive forces are removed, and the insert 16 returns to its original shape to provide secure frictional engagement between the inner surface of the canister 12 with each of the front surface 22, the projections 30, and the feet 38. The design is not reliant on internal geometries (e.g., ribs) associated with the inner surface of canister 12 to couple the insert 16.

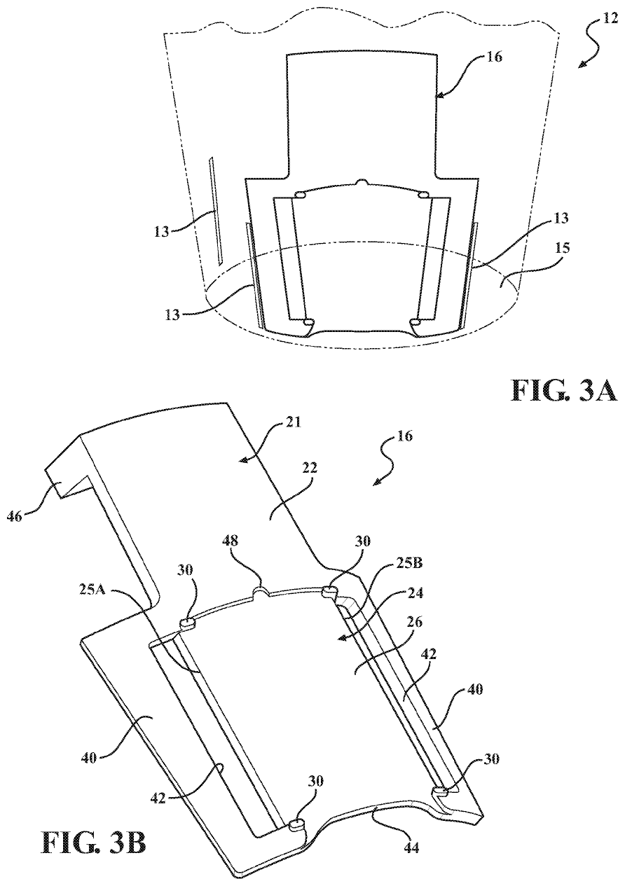

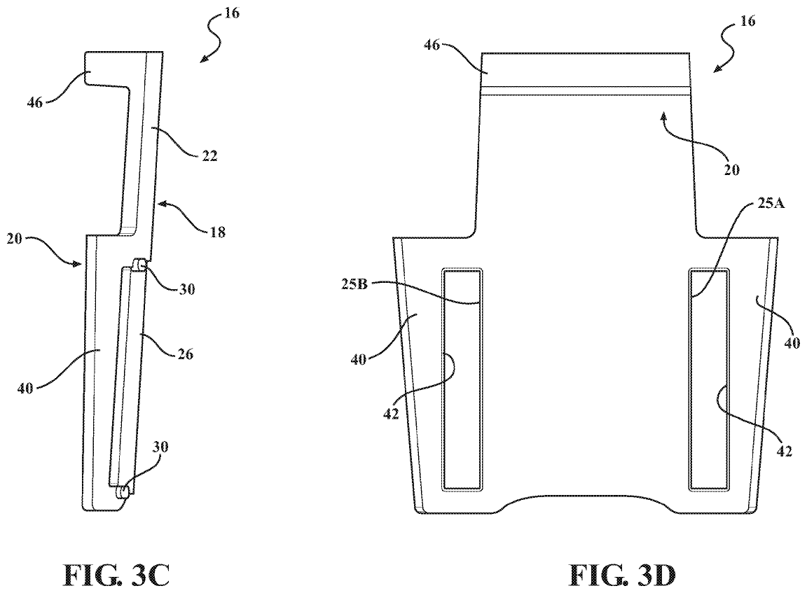

Referring now to FIGS. 3A-5D, the insert 16 may be generally rectangular in shape with a height greater than a width. A thickness of the insert 16 defined between the front side 18 and the rear side 20 is relatively small such that the insert 16 is plate-like in construction. The insert 16 may also be unitary or monolithic in construction. The front side 18 and the rear side 20 may be arcuate with a same radius of curvature as the canister 12. In other words, the front side 18 may be convex and the convexity of the front side 18 is substantially contoured to the inner surface of the canister 12. The shape maximizes a surface area of the front side 18, including the imaging feature 24, for a given volume of the insert 16. With the insert 16 disposed within the canister 12, it minimizes a consumed subvolume of the canister 12 such that volume-based readings are more accurate. The width of the insert 16 is sized to be snugly secured between adjacent ribs 13 formed within the canister 12 (see FIG. 3A). Further, in instances where the ribs 13 are angled inwardly towards the base 15 of the canister 12, the width of the insert 16 may be correspondingly tapered. The taper may help avoid instances where the insert 16 is inverted during assembly, instead being engageable with the ribs 13 of the canister 12 in a single orientation.

The insert 16 includes struts 40 and may define slots 42 between the struts 40. For example, FIG. 3B shows a first strut 40 spaced apart from a first end 25A of the imaging feature 24 to define a first slot 42 therebetween, and a second strut 40 spaced apart from a second end 25B of the imaging feature 42 to define a second slot 42 therebetween. The struts 40 are configured to engage the ribs 13 of the canister 12 to secure the insert 16. Resilient flexibility of the struts 40 and/or the overall insert 16 is configured to facilitate the press-fit interaction with the ribs 13. Further, the resilient flexibility may make the insert 16 usable with canisters of slightly differing dimensions (e.g., ribs spaced closer together or further apart). In certain implementations, the ribs 13 of the canister 12 may be used to couple the insert 16 in an improved manner over existing inserts. The front surface 22 and the imaging feature 24 may be disposed between the struts 40. The front surface 22 and the imaging feature 24 may be generally rectangular in shape with the struts 40 tapering inwardly towards the base 15 of the canister 12. In contrast to the implementation of FIGS. 2A-2D in which the front surface 22 contacts the inner surface of the canister 12, the projections 30 of the present implementation may be disposed adjacent four corners the imaging feature 24. In such an arrangement, the front surface 22 may not directly contact the inner surface of the canister, and therefore fluid may descend along the front surface 22 to be situated in the gap. The slots 42 provide fluid communication from the rear side 20 to the front side 18 of the insert 16. In particular, with the struts 40 being in press-fit contact with the ribs 13 of the canister 12, the slots 42 permit ingress of the waste material into the gap defined between the imaging feature 24 and the inner surface of the canister 12. Certain workflows may indicate agitating or swirling the canister 12 to facilitate homogeneity of the waste material, and the slots 42 encourage a portion of the homogenous waste material to be situated in the gap.

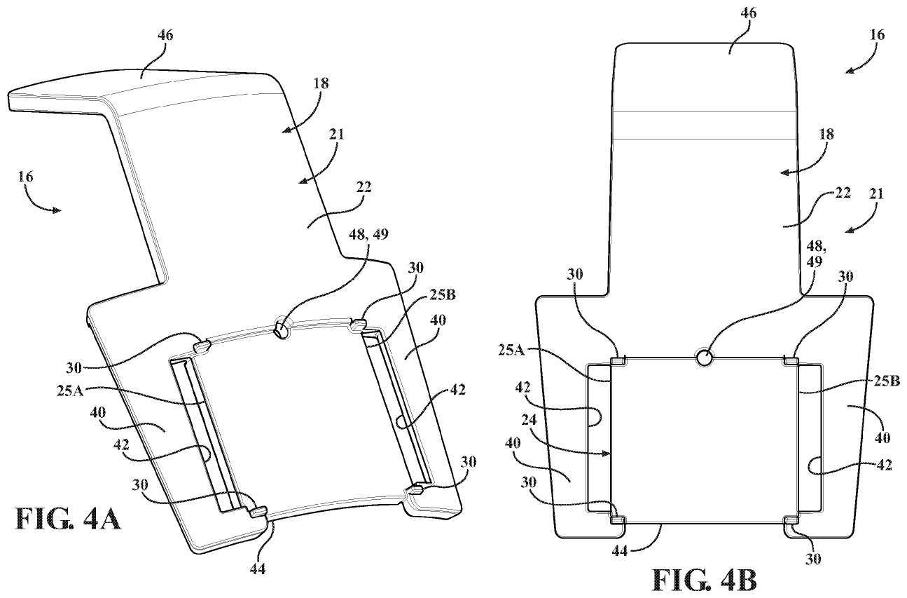

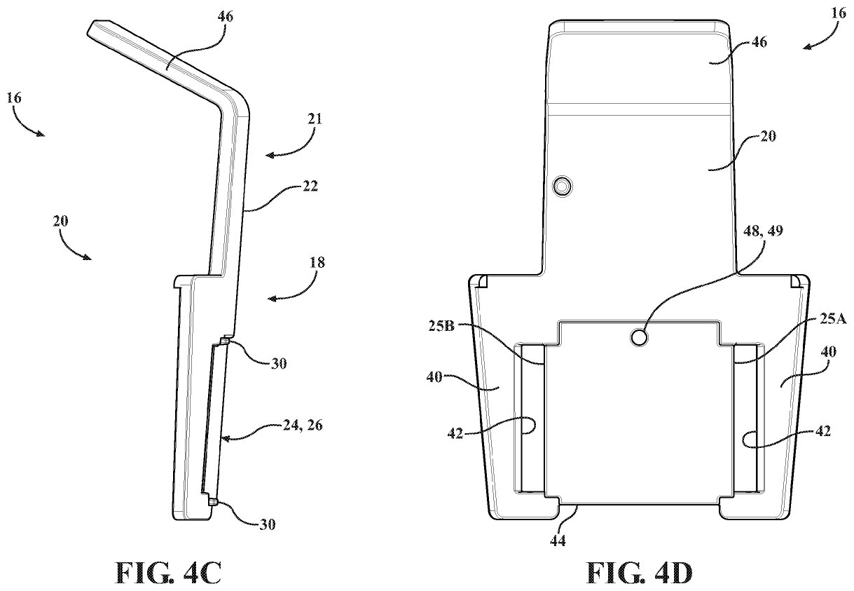

A lower edge of the insert 16 may define a lower vent 44 being similarly configured to promote ingress of the waste material to the gap, particularly at lower volumes of waste material. The insert 16 may also include a handle or grip 46 configured to be manipulated by the user during coupling of the insert 16 with the canister 12. The grip 46 may be a flange extending rearwardly from the front surface 22 or another structure of the insert 16. Depending on the implementation, the grip 46 may be shorter as shown in FIGS. 3A-3B or longer as shown in FIGS. 4A-4D.

The insert 16 may include an alignment feature 48 configured to provide a visual reference to the user when affixing the reference marker 33 to the upper aspect 21, or to the exterior of the canister 12. One example of the alignment feature 48 is a dimple formed on or within the front surface 22 adjacent to the imaging feature 24 as shown in FIGS. 3B and 5A. Another example of the alignment feature 48 is an aperture 49 extending through the insert 16 (i.e., between the front side 18 and the rear side 20, as shown in FIGS. 4A, 4B, and 4D. In addition to providing the visual reference for affixing of the reference marker 33 as described above, the aperture 49 may also permit fluid to flow in and out of the gap formed between the wall of the canister 12 and the insert 16. This fluid flow reduces the formation of air bubbles in the gap, which may otherwise compromise image quality.

In many respects the implementation of FIGS. 5A-5D is similar to those of FIGS. 3A-3D and 4A-4D with like numerals indicating like components. The implementation of FIGS. 5A-5D differs in that the struts 40 extend upwardly in a continuous manner to an upper edge of the insert 16. Further, the insert 16 may include indicia 50 that provides information to the user. The indicia 50 may be a numerical value indicative of the distance of the gap between the imaging feature 24 and the inner surface of the canister 12 with the insert 16 disposed therein. In instances where one of several inserts may be used, the value may be inputted into the user interface for the software to compensate accordingly.

As mentioned, the insert 16 may include the second imaging surface 28. Like the first imaging surface 26, the second imaging surface 28 is recessed from the front surface 22. The first imaging surface 26 is spaced apart from the inner surface by a first distance and the second imaging surface 28 is spaced apart from the inner surface by a second distance. The second distance may be greater than the first distance. The first distance may be within the range of approximately 0.7 to 5.7 millimeters, and more particularly within the range of 1.2 to 3.7 millimeters, and the second distance may be within the range of approximately 1.2 to 6.2 millimeters, and more particularly within the range of 1.7 to 4.2 millimeters.

The second imaging surface 28 provides the aforementioned gradient of increasing color intensity to improve the image-based determinations of the blood component, and further perform additional functionality such as those disclosed in the aforementioned U.S. Pat. No. 9,773,320. It should be appreciated that the insert 16 may include two, three, four, or five or more imaging surfaces with the illustrated implementations being non-limiting examples.

The first imaging surface 26 and the second imaging surface 28 may be positioned lateral to one another in a side-by-side arrangement. As opposed to a vertical arrangement akin to a staircase, the side-by-side arrangement, among other advantages, provides for the color gradient at lower fluid levels. FIGS. 5A-5D show the first imaging surface 26 and the second imaging surface 28 separated by a ridge 52 having a thickness equal to a difference between the first distance and the second distance. For example, the ridge 52 may be approximately 0.5 millimeters. It is contemplated that the ridge 52 may be radiused or otherwise contoured for aesthetics, manufacturability, or function. Other geometries for transitioning between the first imaging surface 26 and the second imaging surface 28 are contemplated.

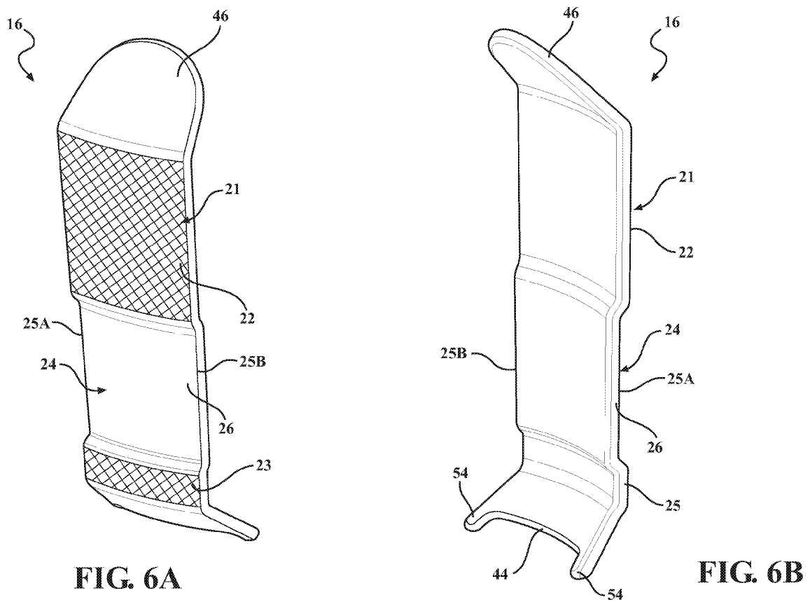

FIGS. 6A and 6B show another implementation of the insert 16 in which adhesive is used to affix the insert 16 within the canister 12. The adhesive may be double-sided tape, for example. The adhesive may be affixed to the front surface 22, and to a lower front surface 23 positioned opposite the imaging feature 24. The adhesive is schematically represented by hatchings. The insert 16 may include the grip 46, and feet 54 of the insert 16 are configured to act as a fulcrum during assembly. More particularly, after removing a fronting of the adhesive(s), the user may pinch the grip 46 and lower the insert 16 into the canister 12 until the feet 54 contact the base 15 of the canister 12 and/or geometries associated with the base 15 of the canister 12. Thereafter, the user moves the grip 46 towards the inner surface to effectively pivot the insert 16 about the fulcrum until the front surface 22 and the lower surface 23 contact the inner surface for the adhesive to secure the insert 16.

It should be appreciated the implementations of the insert 16 discussed herein and the means for supporting the insert 16 within the canister 12 are interchangeable, and more than one means of support may be used on the same insert. For example, the second imaging surface 28, the projections 30, the alignment feature 48, and the indicia 50, among others, may be associated with any of the implementations introduced herein. The insert 16 itself may be formed from a polymer, composite, or other suitable material. As mentioned, the material may be white, opaque, and impermeable to fluid within the canister 12 (see FIG. 2A). The insert 16 may be manufactured from a highly-reflective (i.e., glossy) material and/or coated with a glossy coating. Alternatively, the material may be non-white, non-opaque, and/or fluid permeable. The material and/or the coating may be designed to retard against staining. The insert 16 may be integrally formed through a suitable manufacturing process.

Several implementations have been discussed in the foregoing description. However, the implementations discussed herein are not intended to be exhaustive or limit the invention to any particular form. Modifications and variations are possible in light of the above teachings and may be practiced otherwise than as specifically described. For example, the blood component may be hemoglobin, or otherwise may be one or more of whole blood, red blood cells, platelets, plasma, white blood cells, analytes, and the like. The methods may also be used to estimate a concentration and an amount of a non-blood component within the canister 12, such as saline, ascites, bile, irrigating fluids, saliva, gastric fluid, mucus, pleural fluid, interstitial fluid, urine, fecal matter, or the like.

1. An insert configured to be disposed within a canister for image-based analysis of medical waste to be collected within the canister, the insert comprising:
a front side opposite a rear side;
an imaging feature disposed on the front side and comprising at least one imaging surface configured to be spaced apart from an inner surface of the canister by a gap;
a plurality of projections extending from the front side and configured to engage an inner surface of the canister to maintain the gap between the imaging surface and the inner surface of the canister;
a first strut spaced apart from a first end of the imaging feature to define a first slot therebetween; and
a second strut spaced apart from a second end of the imaging feature to define a second slot therebetween, wherein the first and second struts are configured to engage ribs of the canister to secure the insert within the canister,
wherein the first and second slots extend between the front side and the rear side and are configured to provide fluid communication between the gap and a remaining volume of the canister.

2. (canceled)

3. The insert of claim 1, wherein each of the plurality of projections is positioned adjacent to a respective one of four corners of the imaging surface.

4. An insert configured to be disposed within a canister for image-based analysis of medical waste to be collected within the canister, the insert comprising:
a front side opposite a rear side;
an imaging surface disposed on the front side; and
at least three projections extending forward from the front side and configured to engage an inner surface of the canister to maintain a gap between the imaging surface and the inner surface of the canister.

5. The insert of claim 4, wherein each of the at least three projections is positioned adjacent to the imaging surface.

6. The insert of claim 1, further comprising an upper aspect positioned above the imaging feature and defining a surface sized for a reference marker to be affixed thereto.

7. The insert of claim 6, further comprising an alignment feature positioned adjacent to an edge separating the upper aspect and the imaging feature, wherein the alignment feature configured to be aligned with a complementary geometry of the reference marker.

8. The insert of claim 7, wherein the alignment feature is an aperture extending between the front side and the rear side, wherein the aperture is configured to permit fluid to flow between the front side and the rear side of the imaging feature.

9. The insert of claim 8, wherein the aperture is along a midpoint of a width of the edge.

10. The insert of claim 6, further comprising a handle extending rearwardly from the upper aspect and configured to require insertion of the insert to within the canister in a single orientation.

11. The insert of claim 10, wherein the first and second struts are each tapered inwardly downward so as to be engageable with the ribs of the canister in the single orientation.

12. The insert of claim 1, wherein the imaging feature further comprises a first imaging surface, and a second imaging surface arranged side-by-side to the first imaging surface, wherein the gap is a first gap between the first imaging surface and the inner surface of the canister is different than a second gap between the second imaging surface and the inner surface.

13. (canceled)

14. (canceled)

15. The insert of claim 12, wherein the first gap is within a range of 1.2 to 3.7 millimeters, and the second gap is within a range of 1.7 to 4.2 millimeters.

16. An insert configured to be disposed within a canister for image-based analysis of medical waste to be collected within the canister, the insert comprising:
a front side opposite a rear side;
an imaging feature disposed on the front side and comprising a first imaging surface, and a second imaging surface arranged side-by-side to the first imaging surface; and
a plurality of projections extending from the front side and configured to engage an inner surface of the canister to maintain a first gap between the first imaging surface and the inner surface of the canister, and a second gap between the second imaging surface and the inner surface, wherein the first gap is different than the second gap.

17. (canceled)

18. (canceled)

19. The insert of claim 16, further comprising:
a first strut spaced apart from a first end of the imaging feature; and
a second strut spaced apart from a second end of the imaging feature, wherein the first and second struts are configured to engage ribs of the canister to secure the insert within the canister.

20. The insert of claim 19, wherein a first slot is defined between the first strut and the imaging feature, and a second slot is defined between the second strut and the imaging feature, wherein the first and second slots extend between the front side and the rear side.

21. The insert of claim 19, wherein the first and second struts extend below a lower edge of the imaging feature so as to define a lower vent between the imaging feature and a lower surface of the canister.

22. The insert of claim 16, further comprising an upper aspect positioned above the imaging feature and defining a surface sized for a reference marker to be affixed thereto.

23. The insert of claim 22, further comprising a handle extending rearwardly from the upper aspect and configured to require insertion of the insert to within the canister in a single orientation.

24-28. (canceled)

29. The insert of claim 4, further comprising an upper aspect positioned above the imaging surface and defining a surface sized for a reference marker to be affixed thereto.

30. The insert of claim 29, further comprising an alignment feature positioned adjacent to an edge separating the upper aspect and the imaging feature, wherein the alignment feature configured to be aligned with a complementary geometry of the reference marker.
